(19) United States
(12) Patent Application Publication
(10) Pub. No.: US 2019/0000345 A1
(43) Pub. Date: Jan. 3, 2019

(54) Method And System For Dynamic And Automatic Selection And Configuration Of Processing Or Conditioning Profiles For Characterization Of Physiological Signals

(71) Applicant: General Electric Company, Schenectady, NY (US)

(72) Inventors: Adrian F. Warner, Wauwatosa, WI (US); Daniel R. Schneidewend, Wauwatosa, WI (US); Rodger F. Schmit, Wauwatosa, WI (US); Jennifer L. Johnson, Wauwatosa, WI (US)

(21) Appl. No.: 15/637,249

(22) Filed: Jun. 29, 2017

(51) Int. Cl. A61B 5/0428; A61B 5/00; A61N 1/08; A61B 5/0402; A61B 5/042; A61B 90/90; A61B 90/92; A61B 90/98

(52) U.S. Cl. CPC A61B 5/04288; A61B 5/7203; A61B 5/04282; A61N 1/08; A61B 5/0402; G06F 19/3406; A61B 90/90; A61B 5/6852; A61B 90/92; A61B 90/98; A61B 5/7217; A61B 5/042

(57) ABSTRACT

In the present invention, a configuration system for an electrophysiology (EP) study system provides the physician with the ability to input or select the particular procedure to be performed utilizing the EP system, such as performing an ablation procedure, a pacing procedure, or a diagnostic procedure, among others based on the clinical objective of the procedure. Based on the selection of the procedure to be performed, the EP system can handle the selection and switching of different filter selections for a physiological signal to achieve an optimal signal profile having a clinically acceptable display regardless of acquisition conditions with the minimum of user intervention, or knowledge. These selections may be automatically derived, or manually selected, or over-ridden by the user as needed within any typically, or atypical procedural workflow.

### BACKGROUND OF INVENTION

The invention relates generally to hemodynamic (HEMO), electrophysiological (EP) and other types of recording or mapping devices or systems to which the catheters are connected during studies or monitoring of patients, and particularly to the configuration of the devices and systems for the reduction and/or elimination of noise in the signals to be analyzed.

HEMO/EP devices and systems are used in an increasing number of medical procedures to evaluate various conditions of the patient with which the system is utilized. In many usages of these systems, electrocardiography (ECG) studies record the electrical activity and pathways of a heart to identify, measure and diagnose arrhythmias. In particular, such studies measure electrical changes caused by the depolarization of the heart muscle during each heartbeat. To accomplish this, ECGs utilize electrodes that are combined into channels, the output of which are referred to as a lead.

ECG leads are used in hemodynamic (HEMO) or electrophysiology (EP) studies, which assess electrical activity through the use of catheters placed in the heart through veins or arteries. More specifically, surface ECG leads attached to the patient are used as the reference for the intra cardiac signals from the catheters. That is, they provide a voltage reference to the patient for measurement by other leads.

In this context, ECG leads may encounter noise from a variety of sources such as wireless electrical devices. Moreover, HEMO/EP studies are typically combined with ablation therapy in which a catheter employs radiofrequency energy, for example, to treat arrhythmias. Various medical devices may also attached to a patient during an HEMO/EP study potentially creating noise. In addition, ECG leads have to measure relatively small electrical signals from the patient, less than 20 uV in some instances. As will be appreciated, given the above considerations, achieving acceptable study recordings may be challenging.

To reduce noise, HEMO/EP/ECG systems often utilize a circuit design topology derived from a circuit commonly referred to as “driven right leg” or “right leg drive.” Right leg drive (RLD) circuitry is used to eliminate common mode interference noise and to ensure that recording system ground tracks with the patient. In general, RLD circuits introduce a signal into right leg of a patient to cancel common mode noise from the electrodes. There are currently several RLD circuit topologies that are configured and/or tuned for specific study conditions.

In addition to the RLD, other features of the ECG and/or HEMO/EP system designed to reduce noise include variable gain amplifiers to strengthen the catheter signal, high pass filters to remove signal noise below a certain frequency, low pass filters to remove signal noise above a certain frequency, notch filters to filter signal noise in a preset narrow frequency range or bandwidth, and adaptive filters able to be adjusted to remove signal noise within selectively alterable frequency ranges or bandwidths.

As will be appreciated, however, specific study conditions can often necessitate different noise reduction parameters depending upon the type of procedure being performed and/or the device/catheter being utilized in the procedure for the specific study, among other considerations. In order to accommodate for these changing conditions, the HEMO/EP system can include the capability to adjust the configuration and/or types of noise reduction elements employed for the signals being received by the system. In many cases, the HEMO/EP system is provided with a default configuration for each of the filters associated with the HEMO/EP system. As this default configuration may not be suitable for many types of procedures, each filter type the HEMO/EP system is able to provide multiple settings that can be selected for use depending upon the particular procedure being perform. in this manner, a physician can select the desired configuration for the filter(s) of the HEMO/EP system.

However, due to the large number of combination of the settings or configurations for the filters of the HEMO/EP system, can be very difficult for a physician to have the required knowledge of the HEMO/EP system and all related sources of noise in order to properly configure the filter setting to optimize the noise filtering for the signals received by the HEMO/EP system. For example, in an EP system having five (5) high pass filter settings, seven (7) low pass filter settings and two (2) notch filter settings, this presents a total of seventy (70) possible filter settings for the EP system for the physician to select from for a particular procedure. Additionally, the optimum settings may change during the procedure as different equipment is added or removed.

In certain circumstances, the physician may alter one or more of the filter settings to determine whether the filter setting change increases or decreases the quality of the signal received by the HEMO/EP system. In these situations, the physician can save the settings as a preset filter configuration for the HEMO/EP system which the physician can again select for a subsequent procedure. However, this preset configuration for the filter settings is not optimized for the procedure being performed, as the preset configuration is based on a simple trial-and-error set up performed by the physician. Further, while this preset configuration may be sufficient for a particular procedure, the configuration may not be acceptable for another procedure, thus requiring another trial-and-error process to arrive at a minimally acceptable configuration for that different procedure.

Alternatively, the HEMO/EP system can be preloaded with certain set configurations that are intended for use with different types of procedures. However, these generalized preset configurations are not capable of optimizing the signal noise reduction for a particular procedure.

Further, in other prior art solutions the HEMO/EP system can provide prompts to the user regarding the selection to be made of the filter configuration for the system. One such system is disclosed in U.S. Pat. No. 9,078,578, titled System And Method For Optimizing Electrocardiography Study Performance, and is expressly incorporated by reference herein in its entirety for all purposes. However, in this system, while information is provided to the user regarding an appropriate filter configuration to be employed, the decision on the actual filter configuration to be employed must ultimately be selected by the user.

Accordingly, it is desirable to provide a HEMO/EP system with the capability to automatically and dynamically adjust the configuration of the filter settings for the EP system in order to optimize system performance in a wide variety of study conditions.

### BRIEF DESCRIPTION OF THE INVENTION

There is a need or desire for a HEMO/EP system capable of automatically and dynamically adjusting the noise filtering configuration for the HEMO/EP system in response to the selection of a physiological signal and/or study procedure being performed to obtain the physiological signal to achieve a clinically acceptable display regardless of acquisition conditions with the minimum of user intervention, or knowledge. The above-mentioned drawbacks and needs are addressed by the embodiments described herein in the following description.

According to one exemplary aspect of the invention, an automatic conditioning or filter configuration system is employed on a medical system/computer, such as a HEMO/EP mapping and recording system, in order to automatically configure the filter settings on the HEMO/EP system in response to a selection of a function or procedure to be performed utilizing the HEMO/EP system.

According to another exemplary embodiment of the invention, a configuration system for a hemodynamic (HEMO) or an electrophysiology (EP) study system provides the physician with the ability to input or select the particular procedure to be performed utilizing the HEMO/EP system, such as performing an ablation procedure, a pacing procedure, or a diagnostic procedure, among others based on the clinical objective of the procedure. Based on the selection of the procedure to be performed, the HEMO/EP system can handle the selection and switching of different filter selections for a physiological signal to achieve an optimal signal profile having a clinically acceptable display regardless of acquisition conditions with the minimum of user intervention, or knowledge. These selections may be automatically derived, or manually selected, or over-ridden by the user as needed within any typically, or atypical procedural workflow.

According to another aspect of the invention, a method for selecting an optimized signal profile for an electronic signal monitoring study includes providing an electronic signal monitoring system including an amplifier having a device interface, a controller operatively connected to the amplifier, a plurality of configurable noise filters operatively connected to the controller and amplifier and a signal transmitting device operatively connected to the device interface, receiving information on a type of signal to be applied to the patient through the device and selecting via a program of instructions accessible by the controller an optimal signal profile and associated noise filters for the type of signal to be applied to the patient through the device.

According to a further aspect of the invention, a method for optimizing a return signal in an electronic signal monitoring study includes providing an electronic signal monitoring system including an amplifier having a device interface, a controller operatively connected to the amplifier, a plurality of configurable noise filters operatively connected to the controller and amplifier and a signal transmitting device operatively connected to the device interface, receiving information on a type of signal to be applied to the patient through the device, selecting via a program of instructions accessible by the controller an optimal signal profile and associated noise filters for the type of signal to be applied to the patient through the device and obtaining an optimized return signal through operation of the amplifier using the selected signal profile.

According to still another aspect of the invention, system for configuring an optimal signal profile for an electrophysiology study includes an amplifier having a device interface, a controller operatively connected to the amplifier, a plurality of configurable noise filters operatively connected to the controller and amplifier and a signal transmitting device operatively connected to the device interface for connection to a patient, wherein the controller is configured to automatically select or modify a signal profile of one or more of the noise reduction circuits when provided with information regarding the type of signal to be applied to the patient through the device.

It should be understood that the brief description above is provided to introduce in simplified form a selection of concepts that are further described in the detailed description. It is not meant to identify key or essential features of the claimed subject matter, the scope of which is defined uniquely by the claims that follow the detailed description. Furthermore, the claimed subject matter is not limited to implementations that solve any disadvantages noted above or in any part of this disclosure.

### BRIEF DESCRIPTION OF THE DRAWINGS

The drawings illustrate the best mode presently contemplated of carrying out the disclosure. In the drawings

FIG. 1 is a schematic representation of an EP recording system including a filter configuration system according to one exemplary embodiment of the present invention.

FIG. 2 is a schematic representation of the filter configuration system for the recording system of FIG. 1 according to an exemplary embodiment of the invention.

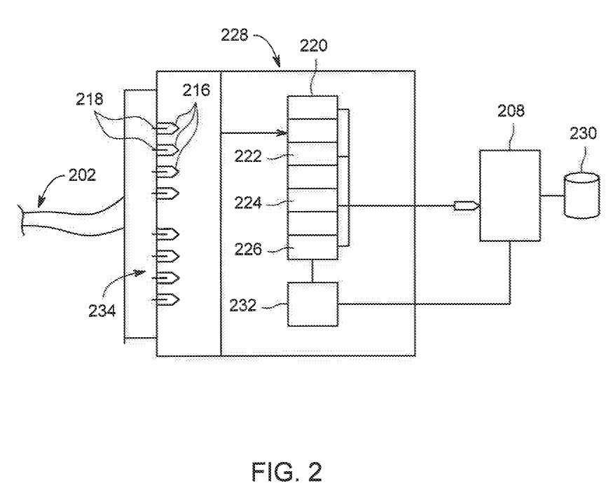

### DETAILED DESCRIPTION OF THE DRAWINGS

In the following detailed description, reference is made to the accompanying drawings that form a part hereof, and in which is shown by way of illustration specific embodiments, which may be practiced. These embodiments are described in sufficient detail to enable those skilled in the art to practice the embodiments, and it is to be understood that other embodiments may be utilized and that logical, mechanical, electrical and other changes may be made without departing from the scope of the embodiments. The following detailed description is, therefore, not to be taken in a limiting sense.

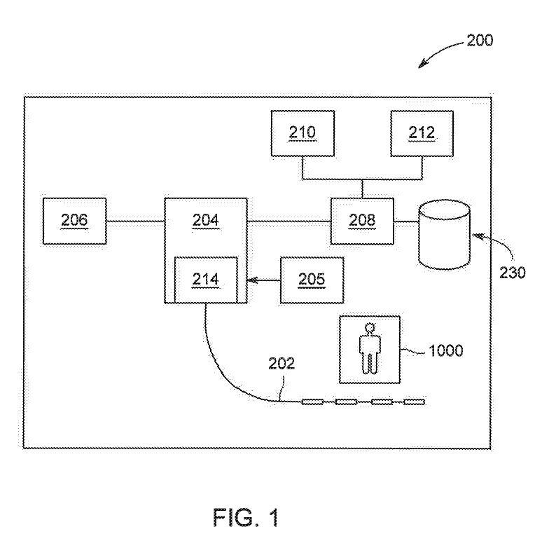

FIG. 1 illustrates one exemplary embodiment of a hemodynamic (HEMO) or an electrophysiology (EP) mapping or an HEMO/EP recorder system 200, such as those used in intracardiac electrocardiography (ECG) studies within the body of a patient 1000. These systems 200 apply an electrical signal (e.g., electrical current) via one or more signal transmitting devices or catheters 202 to various locations of the body of the patient 1000, such as the heart. The system 200 can be similar to that disclosed in US Patent Application Publication No. US2013/0030482, which is expressly incorporated herein in its entirety. In the exemplary illustrated embodiment, the system 200 includes an amplifier 204 that is operably connected between a signal generator 206 and a suitable computer, controller or central processing unit (CPU) 208. In operation, signals generated by the signal generator 206 are transmitted to the catheter 202 by the amplifier 204. A return signal from the patient 1000, such as an ECG signal, is received by the amplifier 204 either via the catheter 202 or another catheter or device 205, and is processed by the amplifier 204 prior to transmitting the return signal to the CPU 208. The CPU 208 performs additional functions on the return signal and displays the information provided by the return signal on one or both of a real-time display 210 and a review display 212.

The amplifier 204 also includes a catheter interface 214 that is used to connect the catheter 202 to the amplifier 204 for use with the recording or mapping system 200. The interface 214 includes a number of pole sockets 216 that are configured to receive corresponding pins 218 disposed on the catheter 202 in order for the catheter 202 to be electrically coupled to the interface 214, and thus enable electric signals to pass between the interface 214 and the catheter 202. The pole sockets 216 are each connected to a catheter signal analog-to-digital converter (ADC) circuit (not shown) and various signal filters within the amplifier 204 in order to convert the analog signals from the pins 218 into digital signals that can be output from the ADC circuit to the CPU 208.

The CPU 208 is operably connected between the amplifier 204 and a number of signal filters 220-226 that can be utilized to condition the return signal from the catheter 202 in order to minimize noise in the return signal, thereby allowing for proper display of the return signal on the displays 210, 212. The signal filters 220-226 include, but are not limited to, a high pass filter 220, a low pass filter 222, a notch filter 224, an adaptive filter 224 and a right leg drive filter 226. Each of the signal filters 220-226 has a number of operational configurations that can be selected to enable the respective filter 220-226 to condition the return signal in a manner that removes unwanted noise from the return signal within the frequencies ranges covered by the respective filters 220-226. The connection of the CPU 208 to the filters 220-226 and to the amplifier 204 enables the CPU 208 to control the settings for each of the filters 220-226 and to alter the gain for the amplifier 204 in order to optimize the return signal that is presented on the displays 210, 212.

The CPU 208 additionally includes a signal configuration system 228. The signal configuration system 228 includes the CPU 208 which is operably connected to a database 230 in which multiple operational configurations or signal profiles for the amplifier 204 and/or filters 220-226 are stored in relation to a particular clinical objective(s) and/or study, function or procedure to be performed using the system 200. The configuration system 228, i.e., the CPU 208, receives input from the user of the system 200 through a user interface 232, such as a keyboard, mouse or touch screen control, in order to determine the function or procedure that is to be performed by the user with the system 200. The CPU 208 can use the information provided by the user to access an appropriate configuration for the amplifier 204 and/or various filters 220-226 that will optimize the return signal to the CPU 208.

In one exemplary embodiment, the configuration system 228 operates by initially providing the user with a set of choices that reference the clinical objective of the study, function and/or individual procedure to be performed. The choices can be provided to the user through the display 212 of the system 200 for ease of reference by the user. For example, the display 212 can enable a user to select from procedures including but not limited to ganglionated plexi, bundle of His or other cardiac feature pacing, surface mapping, stimulation and ablation. Based on the selection from the user, the CPU 208 can access the database 230 and select the configuration or signal profile for the amplifier 204 and/or filters 220-226 that will optimize the return signal for the selected clinical objective of the study, function and/or procedure, thereby maximizing and/or achieving the desired signal acquisition characteristic/signal profile associated with the selected clinical objective. In certain exemplary embodiments, the user can then manually confirm the signal acquisition characteristic/signal profile for use by the system 200.

In determining the signal acquisition characteristic/signal profile, once the user has made the selection of the study, function or procedure to be performed using the system 200, such as by selecting the desired configuration from a drop down menu presented on the display 210, 212 of different studies to be performed using the system 200 and/or types of catheters 202 to be attached to the system 200 for use in a particular study, the filter configuration system 228 will determine the signal acquisition characteristic/signal profile best applicable to the selection. In making this determination, some examples of profile defining characteristics utilized by the system 200 include but are not limited to dynamic range, smallest resolvable signal resolution, expected frequency content of signal, observed frequency content of noise, periodicity of sting/ablation/other inputs, and 3^{rd} party hardware present in signal path, among others, that can be selected relative to the procedural function for that signal channel, in view of the presence of known or unknown or interfering signal(s) in the return signal using a noise identification function or circuit 232 within the configuration system 228. In certain exemplary embodiments, this identification function or circuit 232 for determining the presence of and identifying interfering signal(s) can be automatically performed by the configuration system 228 through the detection and finger printing of noise profiles that may then be used to influence the selection of the signal acquisition characteristic/signal profile. This may be achieved by the noise identification function/circuit 232 through capture of environmental noise, or through quiescent signal analysis and spectral analysis to identify interfering signal content in the return signal. Certain exemplary embodiment of the process and systems for the noise identification function/circuit 232 capable of providing the noise/interfering signal identification or signal noise data to the CPU 208 include but are not limited to those disclosed in U.S. Pat. No. 8,554,311, entitled System And Method Of Noise Reduction In Electrocardiography Study, U.S. Pat. No. U.S. Pat. No. 9,078,578, entitled System and Method For Optimizing Electrocardiography Study Performance each of which are expressly incorporated herein by reference in their entirety for all purposes.

In addition, because the clinical objective can change at any point during the procedure being performed, i.e., can be time dependent, based on an updated selection of the study or procedure currently being performed by the user, or as a result of certain operational changes in the system 200 detected by the CPU 208, such as the switching of the type or characterization of the signal being generated by the signal generator 206, e.g., a switch between a mapping signal and an ablation signal, the CPU 208 can dynamically alter the configuration of the amplifier 204 and/or the filters 220-226 to maintain the optimization of the return signal regardless of the differences in the sensitivity and/or signal to noise ratios, among other characteristics, of the desired signal profile/signal acquisition characteristic. In certain exemplary embodiments, the user can then manually confirm the updated or altered signal acquisition characteristic/signal profile for use by the system 200.

In one exemplary embodiment of the configuration system 228, this dynamic configuration alteration process performed by the configuration system 228 via the CPU 208 can be automated as a result of procedural macro operations or macros, or programming instruction(s) stored within the database 230 relative to a particular study, function and/or procedure. Therefore, at a point in the study, function and/or procedure where the clinical objective is altered, the signal acquisition characteristic/signal profile can be modified accordingly, e.g., to increase noise rejection by changing the configuration of one or more of the filters 220-226, or alternatively increasing fidelity by opening up the signal path aperture within the amplifier 204 to a wide hand at the expense of increased noise in the return signal. In certain exemplary embodiments, the user can then manually confirm the updated or altered signal acquisition characteristic/signal profile for use by the system 200.

In another exemplary embodiment of the configuration system 228, the dynamic automated alteration process performed by the configuration system 228 may be automated by an updated selection of a study, function and/or procedure to be performed using the system 200. For example, in the situation where an ablation, or detection of an ablation signal is selected by the user as the new function to be performed using the system 200/catheter 202 subsequent to performing a diagnostic or mapping function utilizing the system 200 in a study, the signal acquisition characteristic for the ablation requires greater filtration for the duration of this part of the procedure. As a result, the configuration system 228 will operated the CPU 208 to determine the proper configuration for the amplifier 204 and filters 220-226 and automatically place the amplifier 204 and filters 220-226 into the appropriate configuration. In certain exemplary embodiments, the user can then manually confirm the signal acquisition characteristic/signal profile for use by the system 200.

In a further exemplary embodiment of the configuration system 228, as there are different ablation methods that may be utilized in a study, function or procedure, the degree and type of filtering provided to the return signal to provide the desired signal acquisition characteristic/signal profile for the clinical objective of the ablation. For example, the configuration of the amplifier 204 and/or filters 220-226 can be dynamically and automatically tailored by the configuration system 228 to the ablation type or energy being utilized, such as laser, cryogenic, radio frequency or microwave frequency energy, whether the energy type is selected by the user or detected during use by the configuration system 228. Further, some of these types of energy used for ablation might require no change in the signal profile, or might require a general noise reduction strategy/profile which may have been selected globally as a result of other considerations regarding the study, function and/or procedure, as described above regarding the noise identification function/circuit 232.

An important element here is that signal acquisition characteristic/signal profile determined by the configuration system 228 is not a user instruction (UI) for the activation of filter switches. The selection of the signal acquisition characteristic/signal profile can be based on global or single channel requirements, as defined by the study, function and/or procedure selected and the noise present in the signal. So, for example, the configuration system 228 can operate to effect a reduction of general powerline noise introduced by the system 200 in a manner that can be implemented in different ways with different configurations for the amplifier 204 and/or filters 220-226 relative to the signal type being examined, as opposed to a single UI intended to accommodate powerline noise in all types of studies, functions and/or procedures.

Knowledge within the configuration system 228 of any globally disruptive condition obtained by the noise identification function/circuit 232 in any suitable manner described previously can then be addressed in a unique signal-by-signal manner by the configuration system 228 to provide a suitable signal acquisition characteristic/signal profile for each return signal. The automatically and dynamically selected signal acquisition characteristic/signal profile can be overridden by the user if, for example, the physician selects a wide band viewing of a particular return signal. Thus, many different strategies can be employed by the configuration system 228 relative to user requirements, rather than an arbitrary selection of a setting for one or more of the filters 220-226 which may actually confound the clinical objective. The signal acquisition characteristic/signal profile can also be automatically selected when conditions of the acquisition study, function and/or procedure are impacted by stray interfering signals as detected by the noise identification function 232 in any previously described manner. In certain exemplary embodiments, the user can then manually confirm the signal acquisition characteristic/signal profile for use by the system 200.

In one exemplary embodiment of the configuration system 228, the configuration system 228 can automatically select the signal acquisition characteristic/signal profile to include settings for the amplifier 204 and/or filters 220-226 that maintain the subject of interest return signal, while reducing or removing noise/signals that are unwanted, and interfering. In certain exemplary embodiments, the user can then manually confirm the signal acquisition characteristic/signal profile for use by the system 200.

In another exemplary embodiment for the configuration system 228, in light of the detected signal noise, the configuration system 228 can identify a suggested signal acquisition characteristic/signal profile that is best fit or best suited to the current study, function and/or procedure, as initially selected by the user or dynamically determined by the configuration system 228. In certain exemplary embodiments, the user can then manually confirm the suggested signal acquisition characteristic/signal profile for use by the system 200.

In still another exemplary embodiment, the configuration system 228 can utilize a positive determination and characterization of an interfering signal by the noise identification function 232 as input to a heuristic network (not shown) to match the clinical objectives provided by the user with further input from the user as to system variables affecting noise profile such as specific catheters used, 3^{rd} party equipment present in the signal path, patient sedation and medication status, among others.

In addition to the information provided to the configuration system 228 by the user and/or by the noise identification function 232, in another exemplary embodiment the configuration system 228 can additionally receive input directly from the catheter 202 to assist in automatically selecting the proper signal acquisition characteristic/signal profile. In one exemplary embodiment, the catheter 202 is provided with a device/catheter identification system 234, such as that disclosed in US Patent Application Publication No. 2016/0184025, entitled Passive Catheter Identification And Self-Configuration System, the entirety of which is expressly incorporated by reference herein for all purposes. The identification system 234 disposed on the catheter 202 can provide information to the configuration system 228 regarding the type of catheter 202 that is engaged with the system 200, such that the configuration system 228 can utilize that information in addition to the user inputs and information from the noise identification function/circuit 232 in automatically selecting the signal acquisition characteristic/signal profile for the amplifier 204 and/or filters 220-226.

In employing the configuration system 228 and noise identification function 232 within the EP system 200, a number of basic clinical problems are addressed in a dynamic and automatic manner without requiring input from the user, including:
1. selecting settings for an amplifier 204, filter 220-226, or set of filters 220-226 to eliminate noise in the patient physiological signal;
2. providing a grouping of amplifier 204/filters 220-226 relative to the identified noise characteristics of any interfering noise signal(s) to provide a signal acquisition characteristic/signal profile that will result in the optimal signal capture under the conditions of the study, function and/or procedure;
3. identifying the signal acquisition characteristics/signal profile utilizing clinical language rather than a set of filter switch settings;
4. automating the selection and activation of signal acquisition characteristics/signal profiles using macros relative to the current step in the study, function and/or procedure. For example, when switching from diagnostic mode to ablation, the configuration system 228 can automatically and dynamically selects a signal acquisition characteristic/signal profile that deselects settings for the filter 220-226 that may be disturbed by the ablation energy, and selects more appropriate settings for the filters 220-226:
5. providing a signal acquisition characteristic/signal profile including associated filter configurations via the configuration system 228 from the manufacturer, such as by the catheter identification system 234, to support new acquisition, or ablation device types, for example providing a signal acquisition characteristic/signal profile including a 200-220 Hz filter for multi-site, simultaneous burn ablation catheter types such as pulmonary vein ablation catheters (PVAC), multi-array septal catheters (MASC) and multi-array ablation catheters (MARC);
6. providing a signal acquisition characteristic/signal profile including associated filter configurations via the configuration system 228 for devices with varying ablation energy types utilizing knowledge of the particular device type via the catheter identification system 234 and using information interpreted from the macro in the configuration system 228 for the energy type relative to the filter selection to automate selection of the signal acquisition characteristic/signal profile.

The configuration system 228 also provides certain technical advantages, including but not limited to:
1. ensuring the system 200 and catheter 202 are operated with optimal settings relative to the clinical procedure step employed;
2. reduced equipment familiarization required, with the operational language of the system 200 being more procedural versus technical;
3. combinations of filters 220-226 can be combined to solve complex signal acquisition issues for the user, without requiring significant intervention and manual setting of the filter configuration by the user;
4. combinations of filters can be automatically linked via macros stored within the configuration system 228 to allow the user to navigate the procedure actions in any chosen manner;
5. combinations of filters can be applied on a per channel basis allowing the user to optimize visualization relative to the function of the particular channel—for example ablation, Bundle of HIS, etc.

The configuration system 228 also provides certain commercial advantages, including but not limited to:
1. the configuration system 228 decreases physician burden in adjusting setting of the system 200 while performing the procedure;
2. the configuration procedure decreases physician learning time for effective operation of the system 200;
3. the configuration system 228 provides dynamic and automatic adjustment of the signal acquisition characteristic/signal profile and corresponding filter configurations during the procedure to optimize signal characteristics relative to current acquisition conditions, current acquisition task, and per channel set-up;
4. the configuration system 228 can readily align with various noise detection and identification tools, circuits or functions 232 to better support physician debugging during the procedure.

The written description uses examples to disclose the invention, including the best mode, and also to enable any person skilled in the art to practice the invention, including making and using any devices or systems and performing any incorporated methods. The patentable scope of the invention is defined by the claims, and may include other examples that occur to those skilled in the art. Such other examples are intended to be within the scope of the claims if they have structural elements that do not differ from the literal language of the claims, or if they include equivalent structural elements with insubstantial differences from the literal language of the claims.

What is claimed is:

1. A system for configuring an optimal signal profile for an electrophysiology study comprising:
an amplifier having a device interface;
a controller operatively connected to the amplifier;
a plurality of configurable noise filters operatively connected to the controller and amplifier; and
a signal transmitting device operatively connected to the device interface for connection to a patient;
wherein the controller is configured to automatically select or modify a signal profile of one or more of the noise reduction circuits when provided with information regarding the type of signal to be applied to the patient through the device.

2. The system of claim 1 further comprising a user input operatively connected to the controller and through which information regarding the type of signal to be applied to the patient through the device is provided to the controller.

3. The system of claim 1, further comprising a noise identification circuit operatively connected to the controller.

4. The system of claim 3 wherein the noise identification circuit provides signal noise data to the controller so that the controller can assess whether a new signal profile should selected or whether the selected signal profile should be modified.

5. The system of claim 1 wherein the signal transmitting device includes a device identification system that provides information to the controller on the type of signal to be applied to the patient through the device.

6. The system of claim 1 wherein the noise filters are selected from the group consisting of high pass filters, low pass filter, notch filters, adaptive filters, right leg drive circuits and combinations thereof.

7. The system of claim 1 wherein the controller includes a processor and a memory storage database containing a program of instructions allowing the controller to select a circuit profile for the type of signal to be applied to the patient through the device.

8. The system of claim 1 wherein the controller is configured to automatically select or modify the signal profile of one or more of the noise reduction circuits in response to a change in the type of signal to be applied to the patient through the device.

9. The system of claim 1 wherein the controller is configured to automatically modify the signal profile in response to user selected macro operations.

10. A method for selecting an optimized signal profile for an electronic signal monitoring study comprising:
providing an electronic signal monitoring system including an amplifier having a device interface, a controller operatively connected to the amplifier, a plurality of configurable noise filters operatively connected to the controller and amplifier and a signal transmitting device operatively connected to the device interface;
receiving information on a type of signal to be applied to the patient through the device; and
selecting via a program of instructions accessible by the controller an optimal signal profile and associated noise filters for the type of signal to be applied to the patient through the device.

11. The method of claim 10 wherein the step of receiving information on a type of signal to be applied to the patient through the device comprises receiving the information through a user input operatively connected to the controller.

12. The method of claim 10 wherein the step of receiving information on a type of signal to be applied to the patient through the device comprises receiving the information through a device identification system on the device.

13. The method of claim 10 wherein the step of receiving information on a type of signal to be applied to the patient through the device comprises receiving information on a change in the type of signal to be applied to the patient through the device.

14. The method of claim 10 wherein the step of selecting via a program of instructions accessible by the controller an optimal signal profile and associated noise filters for the type of signal to be applied to the patient through the device further comprises operating a noise identification circuit operatively connected to the controller.

15. A method for optimizing a return signal in an electronic signal monitoring study comprising:
providing an electronic signal monitoring system including an amplifier having a device interface, a controller operatively connected to the amplifier, a plurality of configurable noise filters operatively connected to the controller and amplifier and a signal transmitting device operatively connected to the device interface;
receiving information on a type of signal to be applied to the patient through the device;
selecting via a program of instructions accessible by the controller an optimal signal profile and associated noise filters for the type of signal to be applied to the patient through the device; and
obtaining an optimized return signal through operation of the amplifier using the selected signal profile.

16. The method of claim 15 wherein the system further comprises a noise identification circuit operatively connected to the controller, and wherein the method further comprises: modifying the selected signal profile based on signal noise identified by the noise identification circuit.

17. The method of claim 15 wherein the step of selecting the optimal signal profile further comprises:
receiving information on a change in the type of signal to be applied to the patient through the device; and
modifying the selected signal profile based on the change in the type of signal to be applied to the patient through the device.

18. The method of claim 15 further comprising the step of confirming the selection of the optimal signal profile prior to obtaining the optimized return signal.

19. The method of claim 15 wherein the step of selecting the optimal signal profile comprises the steps of adjusting the configuration of one or more of the noise filters.

20. The method of claim 19 wherein the noise filters are selected from the group consisting of high pass filters, low pass filter, notch filters, adaptive filters, right leg drive circuits and combinations thereof.
